(19) United States
(12) Patent Application Publication
(10) Pub. No.: US 2018/0085356 A1
(43) Pub. Date: Mar. 29, 2018

(54) METHOD OF TREATING DISEASE CHARACTERISED BY PROTEIN AGGREGATE DEPOSITION IN NEURONAL CELLS

(71) Applicant: CHRONOS THERAPEUTICS LIMITED, Oxford (GB)

(72) Inventors: Alexandre AKOULITCHEV, Ewelme (GB); Elizabeth MILWAY, Oxford (GB); Elizabeth Jane MELLOR, Kennington (GB); Huw JONES, Royston Hertfordshire (GB)

(21) Appl. No.: 15/723,359

(22) Filed: Oct. 3, 2017

Related U.S. Application Data
(63) Continuation-in-part of application No. 14/893,181, filed on Nov. 23, 2015.

(30) Foreign Application Priority Data
May 24, 2013 (GB) 1309375.2

(51) Int. Cl. A61K 31/436

(52) U.S. Cl. CPC A61K 31/436

(57) ABSTRACT

This invention relates to the use of a very low dose of tacrolimus to treat amyotrophic lateral sclerosis or Parkinson's disease.

This application is a continuation-in-part of U.S. Ser. No. 14/893,181 filed Nov. 23, 2015, which is a U.S. National Stage Entry of PCT/GB2014/051570 filed May 22, 2014, which claims benefit of GB 1309375.2 filed May 24, 2013, the contents of which are incorporated herein by reference in their entireties.

This invention relates to the use of a very low dose of tacrolimus or a close structural analogue to treat a disease characterised by deposition of protein aggregates in neuronal cells. More particularly this invention relates to the use of a very low dose of tacrolimus for the treatment of amyotrophic lateral sclerosis (ALS).

Tacrolimus (also called fujimycin or FK506) is clinically employed as an immunosuppressant, for example, in patients who have had organ transplants, and for the treatment of ulcerative colitis or certain skin conditions. Tacrolimus is available under trade names such as Prograf®, Advagraf® and Protopic®. Commercially available dosage forms of tacrolimus include capsules containing 0.5 mg, 1 mg, 3 mg and 5 mg and ointments for skin conditions where the concentration is 0.05% to 0.19%. Tacrolimus is most commonly administered twice a day for immunosuppression to prevent rejection of transplanted tissues. The clinically employed dose is generally adjusted to produce a whole blood trough concentration of at least 4 ng/mL when seeking to prevent rejection. This is achieved by employing a recommended initial oral dose (two divided doses every 12 hours) which is in the range 0.075 mg/kg/day to 0.2 mg/kg/day which for an average 70 kg patient requires two daily doses of about 2.5 mg to 7 mg. Tacrolimus has also been employed for the treatment of arthritis generally at 3 mg per day. Possibly the lowest dose employed for routine clinical immunosuppression was recorded was for the treatment of myasthenia gravis was 2-3 mg per day (Kanshi et al., J. Neurol. Neurosurg. Psychiatry 2005; 76: 448-450) but this was together with up to 50 mgs per day of prednisolone (and was administered to a lady who may have had low body weight). Clinical trials using low doses of tacrolimus in treating rheumatoid arthritis were described in an editorial in Rheumatology, 2004, 43; 946-948 where in phase II doses of 1 mg, 3 mg and 5 mg of tacrolimus per day were employed and in view of the data from the phase II study, a phase III study was performed using 2 mg and 3 mg per day of tacrolimus.

It is believed that these established clinical uses for tacrolimus operate via a mechanism which acts via calmodulin to activate calcineurin which thus inhibits both T-lymphocyte signal transduction and IL-2 transcription. These are dose dependent mechanisms so that the greater the amount of tacrolimus administered the greater the immunosuppression.

WO 2011/004194 discloses that tacrolimus may be used for the treatment of certain disorders. However, WO 2011/004194 did not disclose that a dose different from conventional clinically relevant immunosuppressant doses of tacrolimus should be employed to treat such diseases.

WO 2000/15208 discloses that tacrolimus may be used for the treatment of certain diseases and notes that the daily dose for chronic use is from 0.1 mg/kg to 30 mg/kg so that for a 70 kg person the daily dose would be 7.5 mg to 210 mg. This range is at least as high as the normal dose range suggested for clinical use of tacrolimus as an immunosuppressant. US 2004/007767 relates to the use of a modified tacrolimus having a methyl group at C₂₁ instead of the propenyl group present in tacrolimus and this also discloses that the daily dose for chronic use is from 0.1 mg/kg to 30 mg/kg.

Gerard et al., J. Neurosciences, 2010, 30(7): 2454-2463 noted that immunophilin ligands including tacrolimus may exhibit neuroprotective effects via inhibition of FKBPs and that the observations validated FKBPs as novel drug targets for Parkinson's disease. Work by others undertaken to develop non-immunosuppressive immunophilin ligands (thereby avoiding undesired effects) was referenced and it was noted that GP1-1485, one such non-immunosuppressive analogue of tacrolimus, did not benefit patients with Parkinson's disease.

WO 2010/056754 disclosed microcapsulated inhibitors of mTOR, especially rapamycin, which could be used for a range of age related disorders. Individual doses were disclosed which were within the range 0.001 mg to 100 mg or even higher and particularised dose ranges of 5 mg/kg to 100 mg/kg were noted. Only effects of rapamycin were exemplified.

Pong et al., Current Drug Targets, 2003, 2: 349-356, disclosed that immunophilin ligands may be considered for the treatment of neurodegenerative diseases. It noted that tacrolimus can inhibit calcineurin and that the mechanism of action of non-immunosuppressive ligands in neuroprotection is unknown. It however noted that attempts had been made to move away from immunogenic molecules and to provide non-immunosuppressant ligands of different structures for use. It further noted that tacrolimus had been shown to be ineffective in the treatment of ALS.

Chattapadhanga et al., Current Medicinal Chemistry, 2011, 18: 5380-5397 discussed the role of neuroimmunophilin ligands and referred to tacrolimus and its C₂₁ ethyl and C₁₈ hydroxyl analogues as first generation ligands. It then described how the skilled person has moved on to second and third generation ligands in the hope of finding effective medicaments for neurodegenerative disorders.

US 2010/0081681 and US 2013/0102569 disclosed that inhibitors of TOR such as rapamycin and analogues may be used to inhibit age related diseases and mentioned the immunosuppressive effects of rapamycin, cyclosporine A and tacrolimus. The experimental data was limited to rapamycin and no suggestion was made that doses could be employed in therapy which were less than the clinically relevant immunosuppressant doses.

It has surprisingly been discovered that very low doses of tacrolimus can provide benefit in the treatment of Amyotrophic Laterial Sclerosis (ALS) and other neurodegenerative diseases, such as Alzheimer's disease, Parkinson's disease, Huntington's disease and other synucleinopathies and tauopathies such as Parkinson's disease dementia and frontotemporal lobe dementia and other dementias and memory loss conditions which may be associated with age related increases of neurotoxic protein aggregation and/or increased oxidative stress or to defect of autophagy. Whilst not wishing to be bound by theory, it is believed that the mechanism by which this neuroprotective effect occurs is multi-modal, which is beneficial since a number of different processes are believed to contribute to neurodegeneration and tacrolimus may be able to interfere with these pathological processes at multiple levels. Tacrolimus is believed to exert its beneficial effects in neurodegenerative diseases via, for example, effects on autophagy and toxic protein accumulation, the oxidative stress response, and neuroinflammation/glial activation.

ALS is a fatal neurodegenerative disease that causes progressive paralysis due to motor neuron death particularly in the motor cortex, spine and brain stem. The disease is often fatal within three years due to atrophy of muscles required for breathing, for example of the diaphragm. Often ALS has a focus in the spinal or bulbar regions of the central nervous system where loss of motor neurons is most pronounced and the loss of motor neurons tends to diminish with distance from that site. ALS is one of the most common neuromuscular diseases worldwide, and people of all races and ethnic backgrounds are affected. ALS has an average global prevalence of 2-7 per 100,000, higher in the UK and USA than many other countries (estimates for the UK are ca. 5,000 ALS patients implying prevalence of over 8 per 100,000). Apart from the clear detrimental effect ALS has on the individual affected and their family, ALS also has an economic impact. These costs can be divided into three components: direct costs, indirect costs and intangible costs. In 2010 the Lewin Group estimated the economic impact of ALS in the US to be $1.03 billion per annum using a moderate prevalence model. The per-patient cost per annum was estimated to be $63,848 and end of life care costs approx. $200,000.

In 90-95% of all ALS cases, the disease occurs apparently at random with no clearly associated risk factors. Individuals with this sporadic form of the disease do not have a family history of ALS, and their family members are not considered to be at increased risk for developing it.

In contrast, about 5-10% of all ALS cases are inherited. This familial form of ALS usually results from a pattern of inheritance that requires only one parent to carry the gene responsible for the disease. Mutations in more than a dozen genes have been found to cause familial ALS. These include the superoxide dismutase SOD-1, the TAR-DNA binding protein TDP-43, and the C9ORF72 open reading frame (see Robberecht and Philips, Nat. Rev. Neurosci. (2013) 14 (4): 248-264).

Despite this diverse etiology of the disease, 97% of patients with ALS display a common phenotype in disease-affected tissues, namely the deposition of the TAR-DNA binding protein (TDP)-43. Deposition of TDP-43 is also the major feature of certain frontotemporal dementias (FTD), associated frontotemporal lobar degeneration (FTLD), which show clinical overlap with ALS.

The role of TDP-43 in ALS is discussed in detail in Scotter et al., 2015, Neurotherapeutics 12(2): 352-363 (the disclosures of which are incorporated herein by reference).

TDP-43, encoded by TARDBP, is a ubiquitously expressed DNA-/RNA-binding protein. TDP-43 contains two RNA recognition motifs, a nuclear localisation sequence (NLS), a nuclear export signal, and a glycine-rich C-terminus that mediates protein-protein interactions. TDP-43 predominantly resides in the nucleus, but is capable of nucleocytoplasmic shuttling. In the nucleus, TDP-43 plays a critical role in regulating RNA splicing, as well as modulating microRNA biogenesis. TDP-43 can regulate the stability of its own mRNA, providing a mechanism for the autoregulation of TDP-43 protein levels. In addition to TDP-43 RNA, TDP-43 regulates the splicing and stability of a large number of other transcripts, and thus influences diverse cellular processes.

Although mostly nuclear, up to ˜30% of TDP-43 protein can be found in the cytoplasm, with nuclear efflux regulated by both activity and stress. TDP-43 is a key component of dendritic and somatodendritic RNA transport granules in neurons, and plays an important role in neuronal plasticity by regulating local protein synthesis in dendrites. TDP-43 is also involved in the cytoplasmic stress granule response—the formation of protein complexes that sequester mRNAs redundant for survival—meaning TDP-43 function is particularly important under conditions of cellular stress.

Many different mechanisms have been proposed to drive ALS pathogenesis. These include, for example, impaired proteostasis, disturbed RNA metabolism, nucleocytoplasmic transport defects, oxidative stress, impaired DNA repair, vesicle transport defects, excitotoxicity, mitochondrial dysfunction, neuroinflammation and astrogliosis, and oligodendrocyte dysfunction (see van Damme et al, Nat. Rev. Neurosci. (2016) Poster: “Molecular Mechanisms of Amyotrophic Lateral Sclerosis”). Tacrolimus has been found to interact with a number of these mechanisms in such a way as to alleviate the development and/or progression of ALS.

The development of therapeutically effective treatments for ALS has proven to present a considerable challenge to the pharmaceutical industry (see Perrin, 2014, Nature 507:423-425, the disclosures of which are incorporated herein by reference).

Over the last decade, about a dozen different experimental treatments have entered clinical trials in patients with ALS. All had previously been shown to ameliorate symptoms or markers of the disease in an established animal model. However, all but one of these experimental treatments failed to show a therapeutic benefit in humans, and the survival benefits in that one (riluzole) are marginal.

Riluzole (Rilutek®, Sanofi) is currently the only approved treatment for ALS; it is a neuroprotective drug that blocks glutamatergic neurotransmission in the central nervous system, thereby preventing apoptosis (programmed cell death) of the motor neuron. No treatments are available in routine clinical use that slow or reverse the progression or disease.

Accordingly, there is a need for improved therapies for the treatment of TDP-43 proteinopathies, such as ALS. The development of efficacious therapies will serve not only to improve quality and longevity of life for those with the disease but will also aid in lowering the cost burden of such diseases.

Although this is of most immediate use in respect of ALS, it is believed that it will also be of benefit for treatment of other diseases where cell damage is associated for the formation of protein aggregates.

The dose of tacrolimus or a close structural analogue thereof employed is less than that used to produce its clinically relevant immunosuppressant effects when treating organ rejection or diseases such as arthritis or myasthenia gravis.

The use of tacrolimus for the treatment purposes described herein is presently preferred over that of a close structural analogue.

Accordingly the present invention provides a method of treating a disease characterised by protein aggregate deposition in neuronal cells which comprises administering to a human in need thereof not more than once a day an effective amount of tacrolimus or a close structural analogue thereof in a dose which does not cause immunosuppression and which produces a trough whole blood level of tacrolimus or its close structural analogue of at least 0.05 ng/mL.

The trough whole blood level may aptly be at least 0.075 ng/mL, for example at least 0.1 ng/mL, such as at least 0.2 ng/mL, or at least 0.3 ng/mL or at least 0.4 ng/mL.

The trough blood level will be less than one quarter that which is considered immunosuppressant when tacrolimus is used as an immunosuppressant in the transplant setting. Generally, this means less than one third of the 4 ng/mL employed in order to prevent transplant rejection i.e. not more than 1.3 ng/mL.

It is believed that to benefit most from the therapeutic window offered by tacrolimus or its close structural analogue the whole blood trough level should be less than 1.2 ng/mL, for example less than 1.1 ng/mL such as less than 1.0 ng/mL.

Aptly the disease to be treated is ALS, Alzheimer's disease, Parkinson's disease, Huntington's disease and other synucleinopathies and tauopathies such as Parkinson's disease dementia and frontotemporal lobe dementia and other dementias and memory loss conditions which may be associated with age related increases of neurotoxic protein aggregation and/or increased oxidative stress or to defect of autophagy (the cell's mechanism for removing damaged cellular components). Each of the above is individually disclosed herein for treatment by this invention. At present it is preferred that each of said diseases are treated using a very low dose of tacrolimus as described herein.

It is presently preferred to employ tacrolimus in this invention. However, it is also apt to employ a close structural analogue of tacrolimus, particularly ascomycin or dihydrotacrolimus. Such analogues include analogues of tacrolimus where the C₂₁ propenyl group is replaced by a methyl group, an ethyl group or a propyl group, or where a C₁₈ hydrogen atom is replaced by a hydroxyl group. Certain of these compounds are less immunosuppressant than tacrolimus, for example the analogue wherein the C₂₁ propenyl group is replaced by a methyl group or the analogue wherein a C₁₈ hydrogen is replaced by a hydroxyl group (for example wherein the C₂₁ propenyl group is unchanged or replaced by a methyl, ethyl or propyl group). U.S. Pat. No. 5,376,663 discloses process for the preparation of analogues of tacrolimus.

The terms “does not cause immunosuppression” and “does not cause clinically relevant immunosuppression” indicate that major side effects of immunosuppression do not normally occur. This results from a dose of tacrolimus or a close structural analogue being employed that does not substantially depress TNFα levels in the patient. It is believed that a dose of less than 1.3 mg per day of such compounds in a 70 kg adult (pro-rata for other body weights) may be considered not to lead to immunosuppression. However, because of individual personal variation, the skilled person will be guided by the blood levels obtained as indicated hereinbefore.

Hence, the maximum daily dose of tacrolimus or a close structural analogue thereof that will be employed for a 70 kg patient will be 1.3 mg (pro-rata for other body weights) and more aptly a dose of not more than 1.0 mg and favourably of not more than 0.9 mg will be employed to a 70 kg patient, for example not more than 0.75 mg (pro-rata for other body weights) will be employed on any day to give a wider separation between the desired effects and side effects.

It is believed that a dose of not less than 0.05 mg per day of tacrolimus or a close structural analogue will be apt and a dose of not less than 0.1 mg per day may be favoured in some cases, for example a dose of not less than 0.15 mg.

Hence, apt daily doses for a 70 kg patient (with doses pro-rata for other weights) include 0.05 mg, 0.075 mg, 0.1 mg, 0.125 mg, 0.15 mg, 0.175 mg, 0.2 mg, 0.225 mg, 0.25 mg, 0.275 mg, 0.3 mg, 0.325 mg, 0.35 mg, 0.375 mg, 0.4 mg, 0.425 mg, 0.45 mg, 0.475 mg, 0.5 mg, 0.525 mg, 0.55 mg, 0.575 mg, 0.6 mg and 0.625 mg, 0.65 mg, 0.675 mg, 0.7 mg, 0.725 mg, 0.75 mg, 0.775 mg, 0.8 mg, 0.825 mg, 0.85 mg, 0.85 mg, 0.875 mg, 0.9 mg, 0.925 mg, 0.95 mg, 0.975 mg, 1.0 mg, 1.025 mg, 1.075 mg and 1.2 mg of tacrolimus or a close structural analogue thereof (for example of tacrolimus). Doses of the active agent are aptly administered orally, for example as a discrete unit dose such as a tablet or capsule. It will be appreciated by persons skilled in the art that doses may be formulated for administration via different routes, including but not limited to topical, ocular, nasal, pulmonary, buccal, parenteral (intravenous, subcutaneous, and intramuscular administration routes.

For convenience of provision of dosing a physician may advantageously wish to employ a fixed dose of tacrolimus across a patient group. Accordingly, provided herein are pharmaceutical compositions for use in treating a condition as described above (and in particular ALS, Parkinson's disease, Alzheimer's disease or Huntington's disease) which comprise 0.05 mg to 0.65 mg tacrolimus, such as 0.1 mg to 0.5 mg tacrolimus, for example 0.15 mg to 0.4 mg tacrolimus, such as 0.2 mg to 0.35 mg tacrolimus and in particular 0.3 mg tacrolimus. Such doses are aptly administered not more than once a day and preferably orally.

Because of the multi-modal mechanism of action of tacrolimus and its close structural analogues by which it provides its beneficial effects in treating disease characterised by protein aggregate deposition in neuronal cells, beneficial results may be obtained by dosing frequency of less than once per day, for example once every 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days or more. Particularly suitable intervals for ease of patient use apart from daily may include on alternative days, once a week, once every 10 days, three times a month, once a fortnight or once a month.

From the preceding commentary on doses, the skilled person will understand that the present invention provides a method of treating a disease characterised by protein aggregate deposition in neuronal cells which comprises administering to a human in need thereof not more than once a day an effective amount of tacrolimus or a close structural analogue thereof wherein the dose is from 0.001 mg/kg to 0.02 mg/kg.

Generally not more than 0.013 mg/kg, for example not more than 0.01 mg/kg will be employed. Aptly not more than 0.0085 mg/kg such as 0.007 mg/kg will be employed.

Generally more than 0.0014 mg/kg, for example more than 0.002 mg/kg will be employed.

Dose ranges that may be mentioned in this respect include 0.0014 mg/kg to 0.0085 mg/kg, for example from 0.002 mg/kg to 0.007 mg/kg of tacrolimus.

It will be appreciated that such doses are very different from doses employed in patients hereinbefore.

In a further aspect the present invention provides a unit dose pharmaceutical composition containing 0.05 mg to 1.3 mg of tacrolimus or a close structural analogue thereof and a pharmaceutically acceptable carrier therefor for use in the treatment of a disease characterised by protein aggregate deposition in neuronal cells.

The disease may be as indicated hereinbefore, for example ALS.

The unit dose may contain not more than 1.2 mg, favourably not more than 0.75 mg, for example a dose of not more than 0.6 mg such as not more than 0.4 mg of tacrolimus or a close structural analogue thereof, preferably tacrolimus.

The unit dose may contain not less than 0.06 mg, favourably not less than 0.1 mg, for example not less than 0.15 mg of tacrolimus or a close structural analogue thereof, preferably tacrolimus.

The tacrolimus or its close structural analogue may be as solvates such as hydrates or alcoholates. Aptly tacrolimus is employed as a hydrate such as the monohydrate (when weights are referred to herein they do not include the weight of the solvating molecule).

Unit doses may contain any of the specific amounts set forth hereinbefore.

At present it is preferred that the unit dose will contain tacrolimus, for example as tacrolimus monohydrate.

If desired an existing commercial product such as Prograf® may be purchased and its contents divided to produce the desired dose which may then be placed into a hard gelatin capsule for oral administration.

The unit dosage form may be liquid, for example a solution or suspension in a container, but it is considered preferable that the unit dose is non-liquid. Suitable solid unit dosage forms include tablets and capsules of which capsules are more apt.

Conveniently, the unit dosage form is suitable for oral administration, for example a tablet or capsule.

In some cases, the unit dosage form may comprise tacrolimus in suspension in a suitable vehicle. Non-limiting examples of vehicles for oral administration include phosphate-buffered saline (PBS), 5% dextrose in water (D5W) and a syrup. The unit dosage form may be formulated to stabilize the consistency of a dose over a period of storage and administration. In some cases, the unit dosage form may contain a solution of tacrolimus or a close structural analogue thereof dissolved in a diluent such as water, saline, or buffers, optionally containing an acceptable solubilizing agent. In favoured form, the composition comprises a solid dosage form. In some cases, the solid dosage form comprises a capsule, a caplet, a lozenge, a sachet, or a tablet. In some cases, the solid dosage form is a liquid-filled dosage form. In some cases, the solid dosage form is a solid-filled dosage form. In some cases, the solid dosage form is a solid-filled tablet, capsule, or caplet. In some cases, the solid-filled dosage form is a powder-filled dosage form. In some cases, the solid dosage form comprises tacrolimus or a close structural analogue thereof in the form of micronized particles, granules or microcapsulated agent. In some cases, the composition comprises an emulsion which may contain a surfactant. In some cases, the solid dosage form comprises one or more pharmaceutically-acceptable excipients, colorants, diluents, buffering agents, moistening agents, preservatives, flavoring agents, carriers, or binders. For example, the solid dosage form may comprise one or more of lactose, sorbitol, maltitol, mannitol, cornstarch, potato starch, microcrystalline cellulose, hydroxypropyl cellulose, acacia, gelatin, colloidal silicon dioxide, croscarmellose sodium, talc, magnesium stearate and stearic acid. In some cases, the solid dosage form comprises one or more materials that facilitate manufacturing, processing or stability of the solid dosage form or a flavoring agent.

Examples of suitable tacrolimus formulations are disclosed in WO2005/020993, WO2005/020994, and WO2008/0145143 and WO2010/005980, the disclosures of which are incorporated herein by reference.

In one embodiment, the unit dosage form comprises a solid dispersion of tacrolimus or a close structural analogue thereof in a dispersion medium comprising a vehicle and a stabilising compound (also referred to as stabilising agent).

It will be appreciated by persons skilled in the art that the unit dosage forms of the invention may be formulated for different routes of administration, including but not limited to oral, topical, ocular, nasal, pulmonary, buccal, parenteral (intravenous, subcutaneous, and intramuscular), vaginal and rectal. Additionally, administration from implants is possible. Suitable preparation forms include, for example, granules, powders, tablets, coated tablets, (micro) capsules, suppositories, syrups, emulsions, microemulsions (defined as optically isotropic thermodynamically stable systems consisting of water, oil and surfactant), liquid crystalline phases (defined as systems characterised by long-range order but short-range disorder; examples include lamellar, hexagonal and cubic phases, either water- or oil continuous), or their dispersed counterparts, gels, ointments, dispersions, suspensions, creams, aerosols, droplets or injectable solution in ampoule form and also preparations with protracted release of active compounds, in whose preparation excipients, diluents, adjuvants or carriers are customarily used.

Formulation strategies for drug delivery of tacrolimus are detailed in Patel et al., 2012, Int. J. Pharm. Investig. 2(4):169-175 (the disclosures of which are incorporated herein by reference).

In one embodiment, the pH in the unit dosage form is below 7 (e.g. as measured by re-dispersion of the composition in water), for example the pH may be in the range from 3.0 to 3.6. The pH may be provided by a stabilizing agent and/or be adjusted by an inorganic or organic acid or a mixture thereof.

Suitable stabilising compounds and stabilising agents for use in a composition of the invention include, but are not limited to, inorganic acids, inorganic bases, inorganic salts, organic acids, organic bases and pharmaceutically acceptable salts thereof.

The organic acid is preferably a mono-, di-, oligo or polycarboxylic acid. Non-limiting examples of useful organic acids are acetic acid, succinic acid, citric acid, tartaric acid, acrylic acid, benzoic acid, malic acid, maleic acid, oxalic acid and sorbic acid; and mixtures thereof. Preferred organic acids are selected from the group consisting of oxalic acid, tartaric acid and citric acid.

The pharmaceutically acceptable salt of an organic acid or inorganic acid is preferably an alkali metal salt or an alkaline earth metal salt. Preferred examples of such salts are sodium phosphate, sodium dihydrogen phosphate, disodium hydrogen phosphate, potassium phosphate, potassium dihydrogen phosphate, potassium hydrogen phosphate, calcium phosphate, dicalcium phosphate, sodium sulfate, potassium sulfate, calcium sulfate, sodium carbonate, sodium hydrogen carbonate, potassium carbonate, potassium hydrogen carbonate, calcium carbonate, magnesium carbonate, sodium acetate, potassium acetate, calcium acetate, sodium succinate, potassium succinate, calcium succinate, sodium citrate, potassium citrate, calcium citrate, sodium tartrate, potassium tartrate, calcium tartrate, zinc gluconate, and zinc sulphate.

Suitable inorganic salts include, but are not limited to, sodium chloride, potassium chloride, calcium chloride, and magnesium chloride.

Stabilised formulations of tacrolimus are described in WO 2011/100975, the disclosures of which are incorporated herein by reference.

Pharmacokinetic analysis was carried out by the Applicant. An oral dose of 2 mg/kg/day tacrolimus in the mouse was found to correspond to a human oral dose of 0.44-0.33 mg/day for a 70 kg person. Mouse studies show that a particularly effective range of doses in a mouse model of ALS is between 0.25 mg/kg/day and 2.5 mg/kg/day. Therefore, even accounting for species differences in pharmacokinetics, preferred oral doses of tacrolimus in humans for the treatment of ALS would be expected to be below 1.3 mg/day and are likely to be below 0.55 mg/day.

In a further aspect the present invention provides a unit dose pharmaceutical composition containing 0.05 mg to 1.3 mg of tacrolimus or a close structural analogue thereof and a pharmaceutically acceptable carrier therefore for use in the treatment of a disease characterized by deposition of protein aggregates in neuronal cells.

Suitably the disease may be ALS. Suitably the disease may be Parkinson's disease. Suitably the disease may be Alzheimer's disease. Suitably the disease may be Huntington's disease.

The unit dose may contain not more than 0.9 mg or 0.75 mg, for example a dose of not more than 0.65 mg such as not more than 0.5 mg or not more than 0.45 mg of tacrolimus or a close structural analogue thereof, preferably tacrolimus.

Such unit dose may be for administration as described herein, particularly for oral administration.

The unit dose may contain not less than 0.05 mg, favourably not less than 0.1 mg, for example not less than 0.15 mg of tacrolimus or a close structural analogue thereof, preferably tacrolimus.

Hence, aptly the unit dose may contain from 0.05 mg to 0.9 mg, for example from 0.1 mg to 0.75 mg, for example from 0.15 mg to 0.6 mg or from 0.15 mg to 0.5 or 0.45 mg of tacrolimus. Such unit doses may be adapted for oral administration, for example as a tablet or preferably a capsule.

It is presently envisaged that the favoured daily dose of tacrolimus for the treatment of diseases characterized by deposition of protein aggregates is from 0.05 to 0.65 mg, for example, 0.1 mg to 0.5 mg, such as 0.15 mg to 0.45 mg, for example, 0.3 mg. Such doses are aptly orally administered, and desirably not more than once per day for example, once a day using the unit doses described herein.

The Examples herein show that the effects of tacrolimus occur in yeast, nematode worms and mammals indicating evolutionary conservation of the molecular mechanisms associated with the amelioraion of neurodegenerative diseases. The Examples show that tacrolimus exerts its effects in the various disease model systems via a multi-modal mechanism, affecting a number of the different pathways that are implicated in the pathology of neurodegenerative diseases such as ALS. These include effects on autophagy and toxic protein accumulation, the oxidative stress response, and neuroinflammation/glial activation. This multi-modal mechanism of action makes tacrolimus a promising candidate for the treatment of neurodegenerative diseases in humans, since a number of different processes are believed to contribute to neurodegeneration and tacrolimus may be able to interfere with these pathological processes at multiple levels. Hence, human disease will be treatable with tacrolimus (and close structural analogues) in the same way as with the other species including the mouse.

### DESCRIPTION OF THE FIGURES

FIG. 1(a) shows the effect of tacrolimus on the chronological lifespan of an ageing S. cerevisiae stationary phase culture grown in the presence of vehicle (DMSO). FIG. 1(b) shows the effect of tacrolimus on the chronological lifespan of an ageing S. cerevisiae stationary phase culture grown in the presence of 40 μM tacrolimus. The viability of the culture is assessed by comparing the growth of 5 μl samples diluted into rich media (“outgrowth cultures”) taken every 1-2 days. The viability of the culture decreases over time in the absence of tacrolimus (FIG. 1(a): successive viability curves shift to the right), but less so in the presence of tacrolimus (FIG. 1(b): successive viability curves are much closer together).

FIG. 2 shows the transcript levels of selected S. cerevisiae genes (as determined by RT-qPCR) in the presence or absence of tacrolimus, and in the presence of an inducer of oxidative stress (hydrogen peroxide; H₂O₂). Comparison of the changes in expression profile suggests that tacrolimus mimics certain aspects of the oxidative stress response.

FIG. 3(a) shows the survival of C. elegans strains that express wild-type human SOD-1 in the presence or absence of tacrolimus. FIG. 3(b) shows the survival of C. elegans strains that express mutant human SOD-1 (127X) in the body wall muscle, in the presence or absence of tacrolimus. Tacrolimus at a concentration of 10 μg/ml in the NGM agar causes a significant increase in lifespan in worms expressing wild-type SOD-1 (p<0.05 for DMSO vs. Tacrolimus, FIG. 3(a)). This increase is virtually abolished when the expression of the autophagy protein encoded by the bec-1 gene is knocked down by RNAi (p>0.05 for bec-1 RNAi DMSO vs. bec-1 RNAi Tacrolimus, FIG. 3(a)), showing that autophagy is required for the effect of tacrolimus to be observed. Similarly in the C. elegans strain expressing mutant SOD-1, tacrolimus causes a significant increase in lifespan (p<0.05 for DMSO vs. Tacrolimus, FIG. 3(b)) and this is virtually abolished when bec-1 expression is knocked down by RNAi (p>0.05 for bec-1 RNAi DMSO vs. bec-1 RNAi Tacrolimus, FIG. 3(b)).

FIG. 4 shows the survival of C. elegans that express human alpha-synuclein in the body wall muscle, in the presence or absence of tacrolimus. Tacrolimus causes a significant increase in lifespan (p<0.05 for DMSO vs. Tacrolimus).

FIG. 5 shows the survival of C. elegans strains that express either a 35 polyglutamine tract fused to YFP (Q35) or YFP alone (Q0) in the body wall muscle. Tacrolimus significantly increases lifespan in both strains (p<0.05 Q0 DMSO vs. Q0 Tacrolimus, p<0.05 Q35 DMSO vs. Q35 Tacrolimus).

FIG. 6(a) shows the effect of tacrolimus on motor neuron neurite network, in a rat motor neuron culture 24 hours after exposure to an Aβ1-42 insult. FIG. 6(b) shows the effect of tacrolimus on motor neuron survival, in a rat motor neuron culture 24 hours after exposure to an Aβ1-42 insult. FIG. 6(c) shows the effect of tacrolimus on extranuclear TDP-43 accumulation (per motor neuron), in a rat motor neuron culture 24 hours after exposure to an Aβ1-42 insult. FIG. 6(d) shows the effect of tacrolimus on caspase-3 levels in a rat motor neuron culture 24 hours after exposure to an Aβ1-42 insult. Tacrolimus causes a concentration-dependent increase in both neurite network (FIG. 6(a): p<0.05 for all concentrations of tacrolimus vs. Aβ) and motor neuron survival (FIG. 6(b): p<0.05 for 100 nM, 1 μtacrolimus vs. Aβ), indicating that tacrolimus has a concentration-dependent neuroprotective effect on motor neurons in this model. Tacrolimus also causes a decrease in the accumulation of extranuclear TDP-43 (FIG. 6(c): p<0.05 for all concentrations of tacrolimus vs. Aβ) and a decrease in caspase-3 levels (FIG. 6(d): p<0.05 for all concentrations of tacrolimus vs. Aβ) indicative of reduced apoptotic activity. (All statistical analyses were carried out vs. Aβ group by one way ANOVA followed by PLSD Fisher's test).

FIG. 7(a) shows that tacrolimus causes a concentration-dependent reduction in glial metabolic activity (p<0.01 for 100 nM tacrolimus+LPS, 1 μM tacrolimus+LPS vs. LPS alone) in a microglial cell culture model of inflammation (stimulated by challenge with 100 ng/ml lipopolysaccharide). FIG. 7(b) shows the presence of reactive oxygen species (p<0.01 for all concentrations of tacrolimus+LPS vs. LPS alone), in a microglial cell culture model of inflammation (stimulated by challenge with 100 ng/ml lipopolysaccharide). FIG. 7(c) shows the microvesicle shedding (p<0.01 for 1 μM tacrolimus+LPS vs. LPS alone) in a microglial cell culture model of inflammation (stimulated by challenge with 100 ng/ml lipopolysaccharide). FIG. 7(d) shows that Tacrolimus also causes a dramatic reduction in the induction of cytokine (IL-6) expression. Therefore tacrolimus significantly reduces the microglial inflammatory response. (All statistical analyses were carried out vs. the LPS alone group by one way ANOVA with Bonferroni as post-hoc test).

FIG. 8(a) shows the rotarod latency of TDP-43 (Q331K) mice (negative control group treated with water) compared with that of non-transgenic control mice (NTg) over 70 weeks (starting at 3 weeks of age). Mice expressing mutant (Q331K) human TDP-43 alone develop a mild but progressive decline in rotarod latency continuing throughout the course of the study. In contrast, the rotarod performance of the non-transgenic control mice remains steady. (n=15 TDP-43 (Q331K) water-treated control (“Water”), n=16 non-transgenic control (“Non Tg”)).

FIG. 8(b) shows the rotarod latency of TDP-43(Q331K) mice treated with Tacrolimus (RDC5) for 70 weeks (starting at 3 weeks of age) at 0.25 mg/kg per day (p.o.), 1.25 mg/kg per day (p.o.) or 2.5 mg/kg per day (p.o.) relative to a vehicle-treated control group. Tacrolimus treatment delays the progression of the decline in rotarod performance at all three doses tested. (n=21 vehicle, n=27 0.25 mg/kg tacrolimus, n=26 1.25 mg/kg tacrolimus, n=19 2.5 mg/kg tacrolimus).

FIG. 8(c) shows the rotarod latency of TDP-43(Q331K) mice treated for 70 weeks with 10 mg/kg riluzole (p.o. per day) relative to a control group treated with water only. Treatment with 10 mg/kg riluzole delays the progression of the decline in rotarod performance. (n=30 10 mg/kg riluzole, n=15 water-treated control).

FIG. 9(a) shows the effect of tacrolimus on amphetamine-induced rotational asymmetry in unilaterally 6-OHDA-lesioned rats. Rats were lesioned at 18 months of age and then treated with tacrolimus (1 mg/kg s.c.) or vehicle on 6 days per week for 6 months. Tacrolimus reduces amphetamine-induced rotational asymmetry relative to the vehicle-treated group, and this effect is significant after 6 months of treatment (p<0.05, unpaired t-test comparing vehicle-treated group vs. tacrolimus-treated group at 6 months). (Baseline: n=23 (vehicle), n=23 (tacrolimus); 2 months: n=22 (vehicle), n=22 (tacrolimus); 4 months: n=17 (vehicle), n=16 (tacrolimus); 6 months: n=11 (vehicle), n=13 (tacrolimus)).

FIG. 9(b) shows the effect of tacrolimus on the accumulation of alpha-synuclein and phospho-Tau (p-Tau) in the brains of unilaterally 6-OHDA-lesioned rats, comparing the number of cells staining positively for each on the contralateral and ipsilateral sides relative to the lesion. On both sides, there is a reduction in the number of cells with visible alpha-synuclein and p-Tau after 3 months tacrolimus treatment relative to the levels in the vehicle-treated group.

The following Examples illustrate the invention.

### EXAMPLE 1

### Effect of Tacrolimus on Age-Related Processes in the Yeast Saccharomyces cerevisiae

### Chronological Lifespan

Analysis of chronological lifespan in yeast involves assessment of the viability of a stationary phase culture over time. Yeast are stored in glycerol stocks at −80° C., inoculated into YPD (Yeast extract-Peptone-Dextrose) and incubated overnight. From this culture, a 1/25 dilution is prepared to produce an ageing culture that is incubated at 30° C. throughout the duration of the experiment. The first 24 hours of yeast growth are assessed in Complete Synthetic Media (CSM) to confirm that the culture has reached stationary phase. After 24 hours, when the culture has completed the exponential growth phase and has entered stationary phase, its viability can then be assessed. Viability is measured by taking a 5 μl sample of the ageing culture and diluting this into a total volume of 150 μlYPD. Growth of this “outgrowth culture” is measured using the Bioscreen-C MBR machine that both incubates the plates and measures optical turbidity every 20 minutes. Comparison of the viability curves of outgrowth cultures taken over several successive days can be used to observe the reduction in viability of the stationary phase culture over time. Specifically, the interval between the time taken for the outgrowth culture to reach OD₆₀₀=0.6 on day 1, and the time taken to reach OD₆₀₀=0.6 on a later day (for example day 7) can be used as a measure of the “ageing rate” of the culture. A greater interval will indicate a higher rate of ageing, i.e. a greater reduction in the number of viable cells. Compounds that delay the rate of ageing (i.e. increase chronological lifespan) will cause the interval to be reduced relative to the equivalent for a control-treated yeast culture.

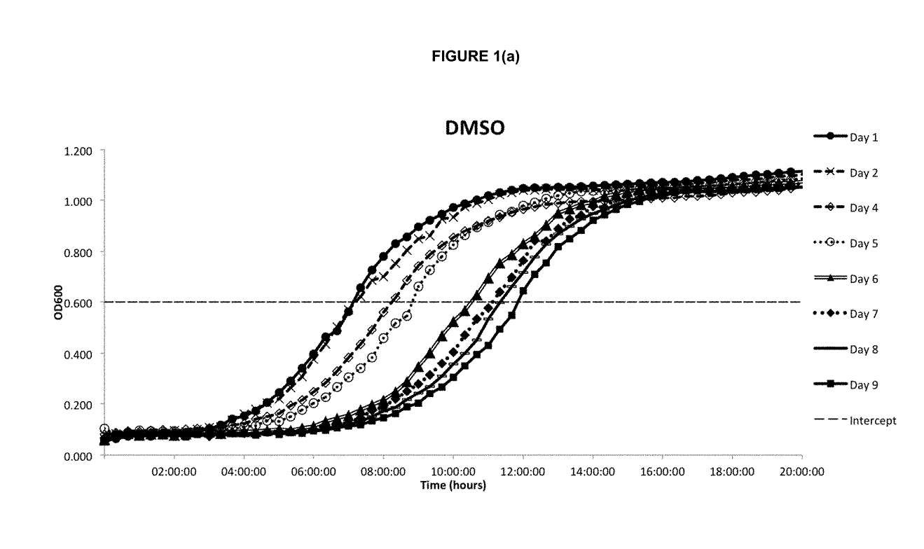

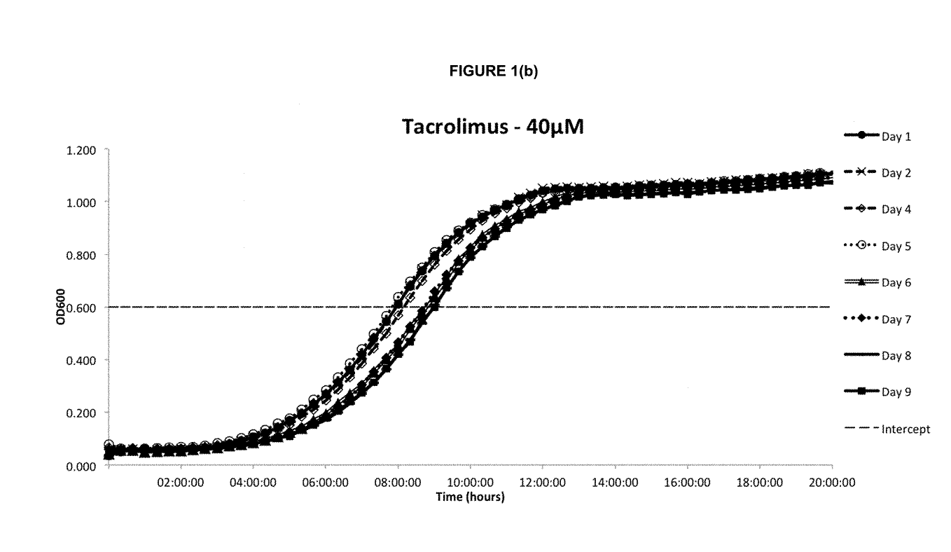

In this way, the chronological lifespan of yeast cultures grown in the presence or absence of 40 μM tacrolimus was compared. FIG. 1(a) shows that, in the absence of tacrolimus, the viability of the culture decreases over time: successive viability curves shift to the right, and the interval between the x-intercepts at OD₆₀₀=0.6 for days 1 and 9 is 287 minutes. However in the presence of 40 μM tacrolimus (FIG. 1(b)), the viability of the culture does not decrease to the same extent: the viability curves on successive days are much closer to that on day 1, and the interval between the x-intercepts at OD₆₀₀=0.6 on days 1 and 9 is only 63 minutes. Therefore 40 μM tacrolimus significantly delays the rate of ageing and extends the chronological lifespan of a stationary phase yeast culture.

### Gene Expression

In order to investigate the potential mechanism(s) by which tacrolimus might delay ageing in yeast cells, the transcription profile of a subset of age/disease-related genes was examined in the presence and absence of tacrolimus.

A pre-culture was diluted 1/100 into synthetic complete medium and tacrolimus was added to a final concentration of 10 μg/ml. (An equivalent volume of DMSO was added to a parallel culture as a control.) The cultures were then allowed to age for 3 days, before RNA extraction and transcript analysis by reverse transcription-qPCR (RT-qPCR). (Transcript levels are normalised to that of the U4 snRNA.)

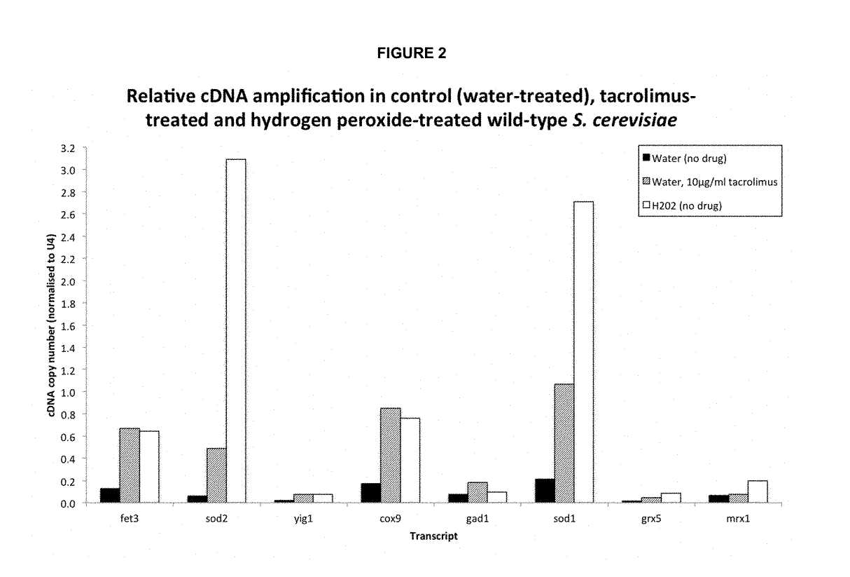

FIG. 2 shows that, in the presence of tacrolimus, the expression of a subset of the analysed transcripts is upregulated. These transcripts include FET3, SOD1, SOD2, and COX9. SOD1 and SOD2 encode superoxide dismutase enzymes, which are important in combatting oxidative stress. FET3 encodes a cell surface ferroxidase involved in iron transport, the expression of which is upregulated in response to DNA replication stress. COX9 encodes a cyctochrome c oxidase subunit involved in the electron transport chain.

Interestingly, when the same experiment was repeated using yeast cultures exposed to hydrogen peroxide for 3 days (but not tacrolimus), the transcript levels for the same four genes also dramatically increased. This suggests that these genes are involved in the oxidative stress response and that treatment with tacrolimus mimics this response, promoting the cell's natural mechanisms for dealing with oxidative stress. As oxidative stress is known to play a role in ageing generally and, more specifically, in neurodegeneration (for example via SOD-1 mutations in familial ALS), this implies that tacrolimus may alleviate age-related processes and neurodegeneration, in part, by promoting cellular defences against oxidative stress.

### EXAMPLE 2

### Effect of Tacrolimus on Neurodegenerative Disease Models in the Nematode Worm, C. elegans

Tacrolimus was tested in a number of disease models using the nematode worm, Caenorhabditis elegans, in which aggregate-prone proteins associated with neurodegenerative diseases are expressed in certain cell types. The effect of tacrolimus was determined by measuring the lifespan of worms in the presence and absence of tacrolimus, using the following general methodology. All of the worms tested using these assays also contained, in addition to the transgene expressing the disease-related protein, a mutation in the bus-5 gene which confers drug-sensitivity.

### Lifespan Assays—General Methodology

C. elegans cultures were synchronised by incubating an asynchronous culture in basic sodium hypochlorite solution to kill everything but the bleach-resistant mature eggs. The eggs were added on to agar plates and then allowed to hatch and develop in the presence of the test compound (or vehicle) and bacterial food source. When the worms reached the penultimate larval stage (L4; immediately prior to development of the next generation of mature eggs), they were transferred to fresh growth plates containing 5-fluoro-2′-deoxyuridine (FUDR) and the test compound (or vehicle), along with the bacterial food source. FUDR was added to prevent further egg maturation and hatching to prevent interference from development of subsequent worm generations. Thereafter the worms were allowed to age in the presence of the test compound/vehicle and FUDR. From this point on, each ageing population was inspected at regular intervals (e.g. daily) and viability assessed by counting the number of dead worms.

The cumulative number of dead worms for each population was used to generate a survival/viability plot to determine chronological lifespan (CLS). Survival was analysed using the Kaplan-Meier log-rank survival analysis method, using the Online Application for Survival (OASIS; see http://sbi.postech.ac.kr/oasis). The principle of the analysis method is that the proportion of dead worms on each day is related to the size of the ‘at risk’ population (i.e. the number of remaining live worms), which declines as the number of worms in the study continues. By comparing the ‘observed’ number of dead worms in the treatment group against the ‘expected’ number of dead worms in the ‘at risk’ group (the treatment+control groups are combined as the best estimate of the ‘at risk’ group), it is possible to determine the significance of any differences between the ‘observed’ and ‘expected’ deaths using the Chi-Squared test. The cumulative Chi-Squared probability over all the days of the study then indicates whether there is a significant difference between the control and treatment groups (a Chi-Squared p-value of <0.05 is accepted as a significant effect).

### Amyotrophic Lateral Sclerosis—SOD-1 Toxicity

Mutations in the human SOD-1 gene are associated with familial ALS, and the aggregation of both mutant and wild-type forms of SOD-1 has been observed in the motor neurons of ALS patients (Robberecht and Philips, Nat. Rev. Neurosci. (2013) 14 (4): 248-264).

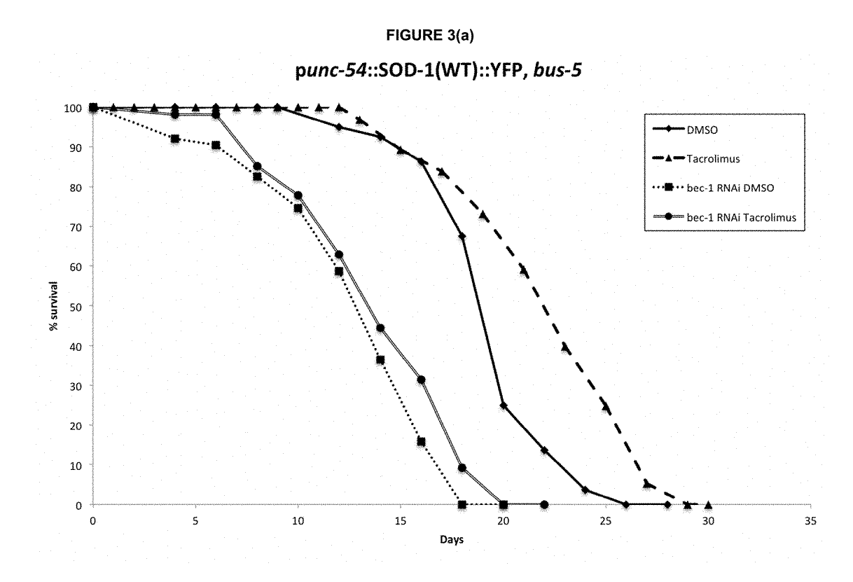

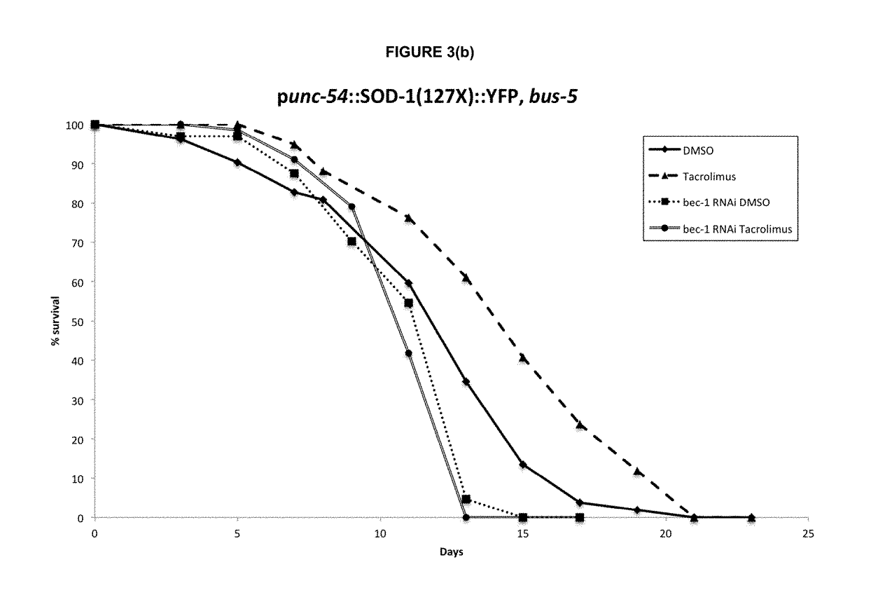

The effect of tacrolimus on C. elegans expressing wild-type or mutant human SOD-1 in the body wall muscle was determined. Note that the lifespan of worms expressing mutant SOD-1 (127X) was shorter than that of worms expressing the wild-type version (compare DMSO curves in FIGS. 3(a) and 3(b)), consistent with the association between mutant SOD-1 and familial ALS.

Tacrolimus significantly increased the lifespan of both C. elegans expressing wild-type SOD-1 (FIG. 3(a)) and C. elegans expressing mutant SOD-1 (FIG. 3(b)). Note that in both cases, the effect was virtually abolished when the expression of the bec-1 gene was knocked down by RNAi (worms were grown in the presence of bacteria expressing double stranded bec-1 RNA, rather than the standard bacterial food source). The bec-1 gene encodes the C. elegans ortholog of mammalian autophagy proteins Atg6/Vps30/Beclin1; by homology, BEC-1 may be part of a Class III phosphatidylinositol 3-kinase complex that plays a role in localizing autophagy proteins to preautophagosomal structures. Therefore one mechanism by which tacrolimus may be acting in order to ameliorate the effect of the SOD-1 expression is via autophagy.

### Parkinson's Disease—Alpha-Synuclein Toxicity

Alpha-synuclein aggregation is implicated in the pathology of Parkinson's disease. C. elegans that express alpha-synuclein in the body wall muscle form alpha-synuclein inclusions as the worms age and have a reduced lifespan (van Ham et al, PloS Genet. (2008) 4 (3): e1000027).

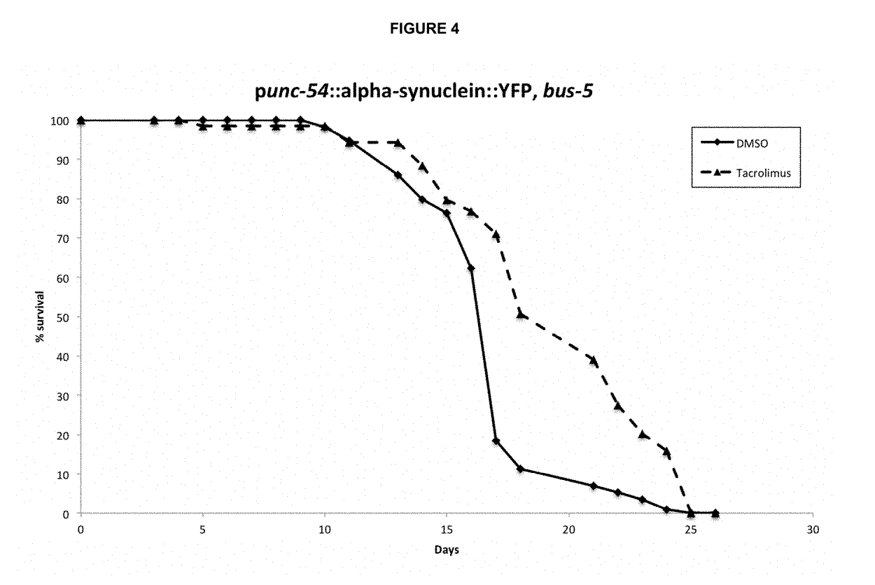

Tacrolimus ameliorates this effect by significantly increasing the lifespan of alpha-synuclein-expressing worms, as shown in FIG. 4.

### Huntington's Disease—Polyglutamine (PolyQ) Toxicity

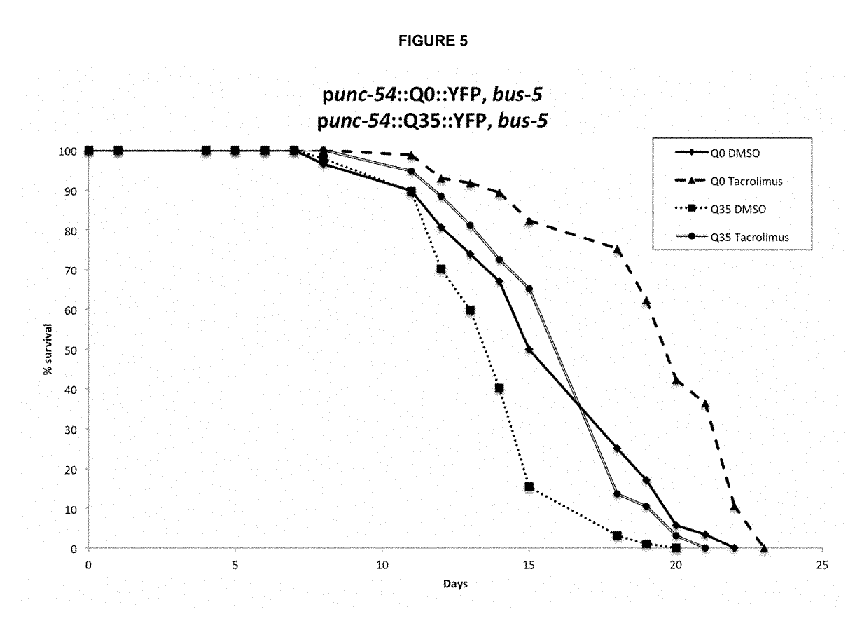

Triplet repeat expansions which, when expressed, translate into extended tracts of polyglutamine residues (polyQ) are a feature of a number of diseases, including Huntington's disease in which a polyQ tract is expressed within the huntingtin gene. The expression of a polyQ repeat sequence containing 35 glutamine residues in the body wall muscle of C. elegans causes the formation of aggregates and a decrease in motility (Morley et al, PNAS (2002) 99 (16): 10417-10422). There is a corresponding decrease in lifespan, which is ameliorated by the presence of tacrolimus (FIG. 5).

Therefore tacrolimus consistently reduces the effect of disease-associated protein expression on lifespan in a number of C. elegans models of neurodegenerative disease, indicating that it can counteract at least some of the cytotoxic effects of these proteins. Therefore it would also be expected to counteract the cytotoxic effects of these proteins when aberrantly expressed in the neurons of human patients.

### EXAMPLE 3

### Effect of Tacrolimus on a Cell Culture Model of TDP-43 Toxicity in Motor Neurons

Primary motor neuron (MN) cultures generated from rat spinal cord were treated with 20 μM amyloid beta 1-42 peptide (Aβ1-42). This insult causes an acute increase in TDP-43 levels in the cells and serves as a model for the TDP-43 mislocalisation, aggregation and toxicity observed in the motor neurons of ALS patients (reviewed in Callizot et al, poster “Amyloid peptide and cytoplasmic TDP-43 accumulation in pathogenesis of ALS: an in vitro study”, http://www.neuro-sys.fr/IMG/pdf/poster_adpd2017_tdp43.pdf). This model was used to determine the effect of tacrolimus on TDP-43 toxicity in motor neurons.

After 8 and 24 hours following the Aβ1-42 insult, the cell culture supernatant was taken off and the MN culture was fixed by a cold solution of ethanol (95%) and acetic acid (5%) for 5 minutes at −20° C. After permeabilisation with 0.1% saponin, one group of cells was incubated for 2 hours with a) mouse monoclonal anti-microtubule-associated-protein 2 (MAP-2) antibody, and b) rabbit polyclonal anti-TDP-43 antibody. The anti-MAP-2 antibody was used to determine MN survival (number of stained MN) and MN neurite length. The anti-TDP-43 antibody was used to examine extranuclear TDP-43: the area of extranuclear TDP-43 staining per MN was calculated by normalising to the number of MAP-2 positive cells.

A separate group of cells was incubated with a) mouse monoclonal anti-microtubule-associated-protein 2 (MAP-2) antibody, and b) rabbit polyclonal anti-caspase-3 antibody. This group was used to determine the number of caspase-3-positive MN, i.e. the number of overlapping MAP-2 and caspase-3-positive cells.

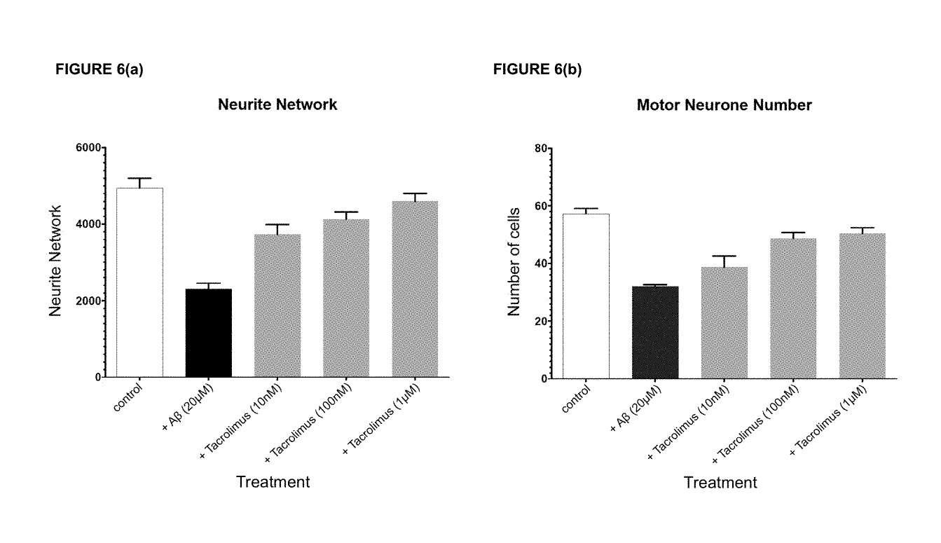

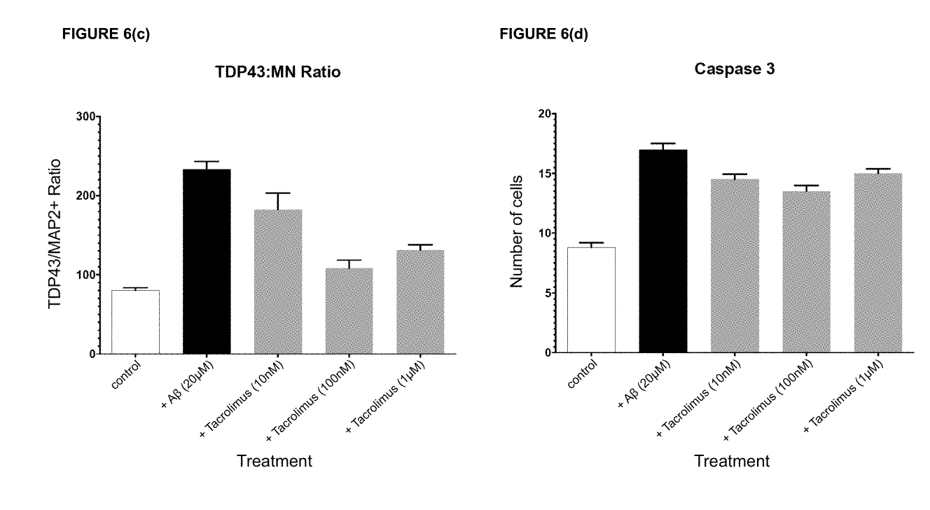

At 8 hours following the Aβ1-42 insult, TDP-43 levels were significantly elevated (data not shown) but there was no disruption to motor neuron number or network. However after 24 hours, there was a significant loss of neurite network (reduced by 53%) (FIG. 6(a)) and a significant decrease in motor neuron number (reduced by 44%) (FIG. 6(b)). Cells that had been treated with tacrolimus at 3 different concentrations 1 hour before Aβ1-42 application showed a significant reduction in the loss of neurite network (FIG. 6(a)) and motor neuron number (FIG. 6(b)), indicating that tacrolimus has a dose-related neuroprotective effect in this model. Note that the effect of tacrolimus is comparable to that of the only currently licensed drug for treatment of ALS, riluzole, when tested at 1 μM and 5 μM (10 μM riluzole was toxic to the cells).

Extranuclear TDP-43 accumulation was also reduced by tacrolimus. In the control cells, extranuclear TDP-43 levels (normalised to motor neuron number) were increased by 191% 24 hours after the Aβ1-42 insult, relative to the levels in cells that were not challenged with Aβ1-42. However the accumulation of TDP-43 was reduced in cells treated with tacrolimus. When normalised to motor neuron number, all 3 concentrations of tacrolimus significantly reduced TDP-43 levels (TDP-43:MN ratio in FIG. 6(c)), with the greatest effect at 100 nM: at this concentration, the TDP-43:MN ratio was only increased by 35% in response to the Aβ1-42 insult (relative to the unchallenged cells). This is better than the reduction observed for the current standard of care, riluzole: the greatest effect of riluzole was observed at 5 μM, at which the TDP-43:MN ratio increased by 51% in response to the Aβ1-42 insult.

FIG. 6(d) shows the induction of caspase-3 in the same cells, in response to Aβ1-42 insult. Caspase-3 is a key component of the apoptotic cell death pathway: it is activated in response to both extrinsic and intrinsic cell death signals, and is a key component of neuronal death in, for example, Alzheimer's disease. In response to Aβ1-42 insult, caspase-3 levels are increased by 91% relative to levels in the unchallenged cells. However the increase was less in cells that were pretreated with tacrolimus at all 3 concentrations, with the greatest effect at 100 nM. This indicates that the level of caspase-3 induction, and hence apoptotic cell death, is reduced by the presence of tacrolimus. This compares favourably with the effect of riluzole at 1 μM and 5 μM.

Overall the results of the motor neuron cell culture experiments indicate that tacrolimus is neuroprotective, reduces the induction of TDP-43 and reduces the induction of apoptotic cell death in response to an external insult (Aβ1-42). The timing of the effects indicates that the earliest step in the response to the Aβ1-42 insult (which leads eventually to motor neuron cell loss) is the accumulation of TDP-43, as this can be observed after 8 hours. The observation that tacrolimus can reduce the accumulation of TDP-43 indicates potential efficacy as a treatment for ALS, as TDP-43 accumulation is associated with 97% of ALS cases.

### EXAMPLE 4

### Effect of Tacrolimus on a Glial Cell Culture Model

Glial cell activation and a sustained neuroinflammatory response is commonly found in the spinal cords of ALS patients and is believed to contribute to the degeneration of motor neurons (Lee et al, Exp. Neurobiol. (2016) 25 (5): 233-240). In particular, there is increasing evidence that chronic activation of microglia, for example via cellular stresses associated with ALS, may lead to non-cell autonomous motor neuron damage.

To investigate the effect of tacrolimus on microglial function, rat microglia were isolated as follows. A primary glial co-culture was isolated from P2 Sprague Dawley rat pups. The cells were cultured until confluency and then the microglia were isolated from the confluent glial cells through mechanical shaking. The isolated microglial cells were then subjected to inflammatory challenge by overnight exposure to 100 ng/ml lipopolysaccharide (LPS), in the presence or absence of tacrolimus. Glial cell activity was monitored by determining cell viability (Cell Counting Kit-8), the presence of reactive oxygen species (by quantitative fluorescence measurement in the supernatant/cell lysate), microvesicle shedding (by monitoring vesicle formation in cells loaded with Calcein-AM and then challenged with 1 mM ATP) and inflammatory cytokine production (by qPCR).

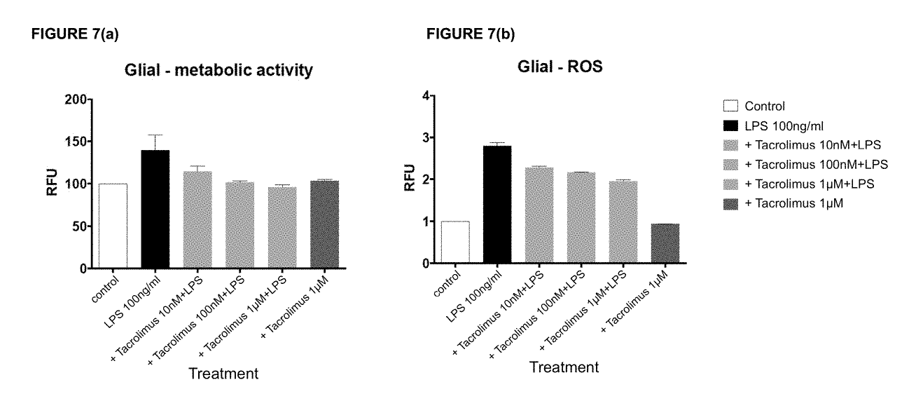

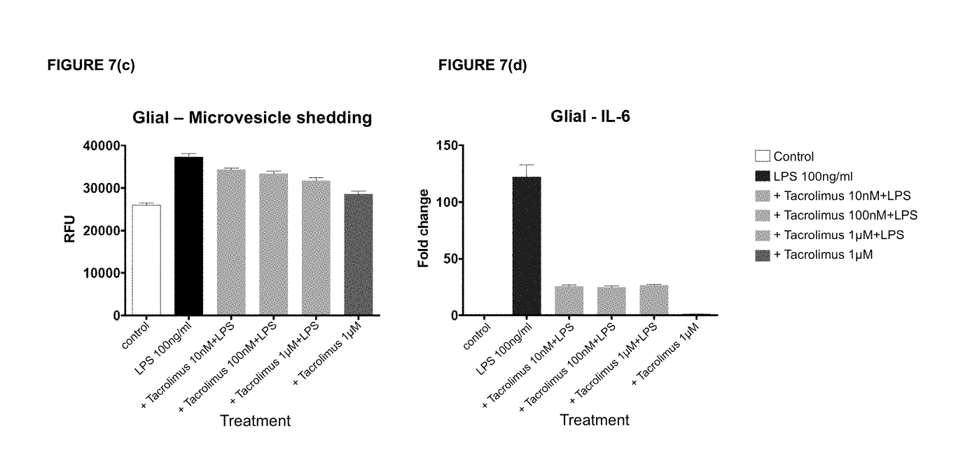

The addition of LPS causes a significant increase in the metabolic activity of glial cells (FIG. 7(a)). However the presence of tacrolimus reduces this increase in a concentration-dependent manner.

Similarly, LPS also causes a significant increase in oxidative stress in glial cells (as monitored by the presence of reactive oxygen species) (FIG. 7(b)). This is significantly reduced by the presence of tacrolimus, in a concentration-dependent manner.

FIG. 7(c) shows that LPS induces a significant increase in microvesicle shedding, indicative of microglial activation: again, this increase is reduced in the presence of tacrolimus in a concentration-dependent manner.

Finally, FIG. 7(d) shows the effect of LPS and tacrolimus on glial cytokine production. LPS causes a dramatic increase in the expression of IL-6. The induction of this cytokine is significantly reduced in the presence of tacrolimus.

Collectively, these assays demonstrate that tacrolimus significantly reduces the activation of microglia in response to an inflammatory challenge. Since neuroinflammation is believed to play a key role in motor neuron damage in ALS, this indicates that tacrolimus could ameliorate the neuronal damage caused by glial cell activation in ALS patients.

### EXAMPLE 5

### Effect of Tacrolimus in Mice Expressing WT and/or Q331K TDP-43

### Mice

Transgenic mice expressing wild-type human TDP-43 or human TDP-43 carrying a point mutation (Q331K) have been developed and described by the Shaw lab (see Arnold et al., 2013, Proc. Natl. Acad. Sci. 110(8):E736-45 and Mitchell et al., 2015, Acta. Neuropathol. Commun. 3(1):36; the disclosures of which are incorporated herein by reference). The inserted constructs placed the cDNA for N-terminal myc-tagged wild-type or mutant TDP-43 under the control of the mouse prion promoter, resulting in expression in the CNS. Because the constructs do not contain the 3′UTR of the human TDP-43 gene, TDP-43 mRNA levels are not autoregulated and therefore TDP-43 levels in the transgenic strains reach 2-3 fold above endogenous levels.

Hemizygous lines for each construct were established (TDP-43(WT) and TDP-43(Q331K) respectively), and crossing these lines produced compound hemizygous animals (TDP-43(WTxQ331K)).

For this study, single transgenic mice were generated from existing mouse lines and genotyped (by PCR of DNA extracted from tail-tip samples) before reaching 3 weeks of age. Young breeding pairs (approx. 6-8 weeks old) of TDP-43(WT) and TDP-43(Q331K) were established to generate mice co-expressing both transgenes (TDP-43(WTxQ331K), together with single WT, Q331K and non-transgenic (NTg) littermates. Only the single TDP-43(Q331K) and double (TDP-43(WTxQ331K)) transgenic animals were required for this study. However, the double (TDP-43(WTxQ331K)) transgenic animals rapidly developed a disease phenotype (as previously described: see Mitchell et al., 2015, Acta. Neuropathol. Commun. 3(1):36) and did not survive long enough for dosing to commence. Therefore, only the single (TDP-43(Q331K)) transgenic animals were used to test the efficacy of tacrolimus or riluzole.

Multiple breeding rounds were required to achieve sufficient numbers of animals per treatment group and dosing/testing were staggered accordingly. All animals were tail-tipped for genotyping and ear-punched for identification at 2 weeks of age. Litter-mates were randomly distributed into each treatment group where possible (see below), with the aim of achieving equal numbers of males and females in each treatment group.

### Drug Treatment

Following a 2-day recovery period after tail-tipping, mice were acclimatised with a polypropylene oral gavage (delivering water only) each morning for 5 days. At 3 weeks of age (following the 5-day acclimatisation period) drug dosing commenced. Dosing was carried out 6 days a week by oral gavage (in a volume of 5 ml/kg), with the following treatment groups:
Water, negative control
Vehicle (1% Cremophor RH40; 4% dehydrated ethanol (w/v)), negative control
Tacrolimus (2.5 mg/kg)
Tacrolimus (1.25 mg/kg)
Tacrolimus (0.25 mg/kg)
Riluzole (10 mg/kg in water), positive control

Dosing was continued for 70 weeks.

Based on pharmacokinetic analysis, an oral tacrolimus dose of 2 mg/kg/day in the mouse is believed to correspond to a human oral dose of about 0.33-0.44 mg/day for a 70 kg person.

Riluzole is currently the only FDA-approved drug for treatment of ALS and was therefore used as a positive control to confirm the validity of the model.

### Behavioural and Phenotypic Assays

Mice were weighed after 3 days of oral gavage acclimatisation (2 days prior to the start of dosing), and thereafter on a weekly basis beginning on day 0 (the day before dosing commenced).

Mice were assessed by rotarod testing on a weekly basis. Each animal was acclimatised and trained on the rotarod 2 days prior to the start of dosing, by first undergoing a basic 2 minute acclimatisation at 5 rpm, and then undergoing 2×5 minute training sessions using a 2-20 rpm acceleration paradigm. On the following day (the day before dosing commenced, day 0), each animal was tested using a 5 minute 2-30 rpm paradigm to give a baseline reading. Thereafter, testing was conducted on a weekly basis (on day 7, 14, etc.) using a single 5 minute 2-30 rpm paradigm. Testing was conducted at the same time each afternoon.

General animal health and welfare was monitored throughout the dosing period and any unusual phenotypes or behaviours were recorded.

### Results and Conclusions

### Tacrolimus Delays the Decline in Motor Function in TDP-43(Q331K) Mice

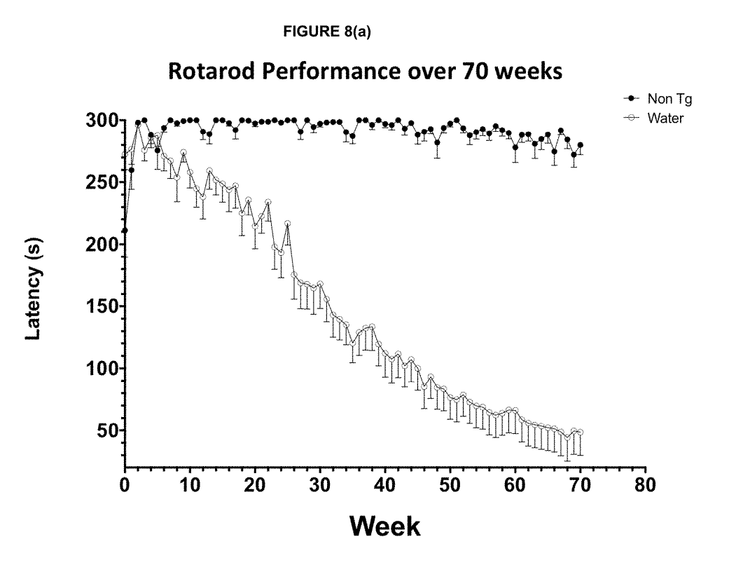

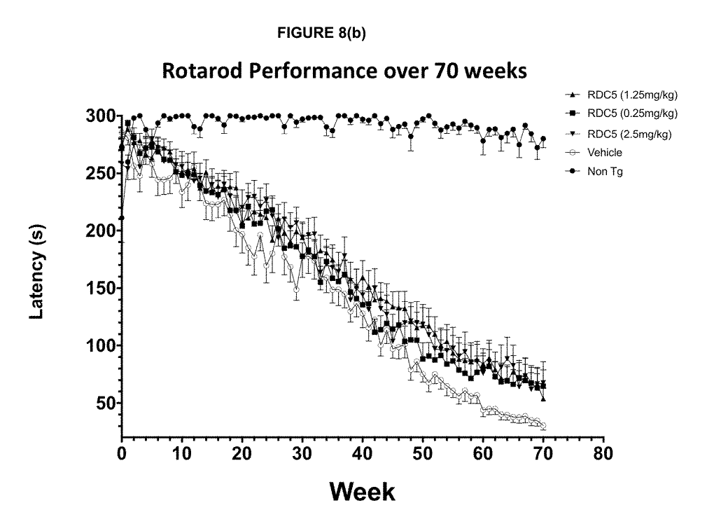

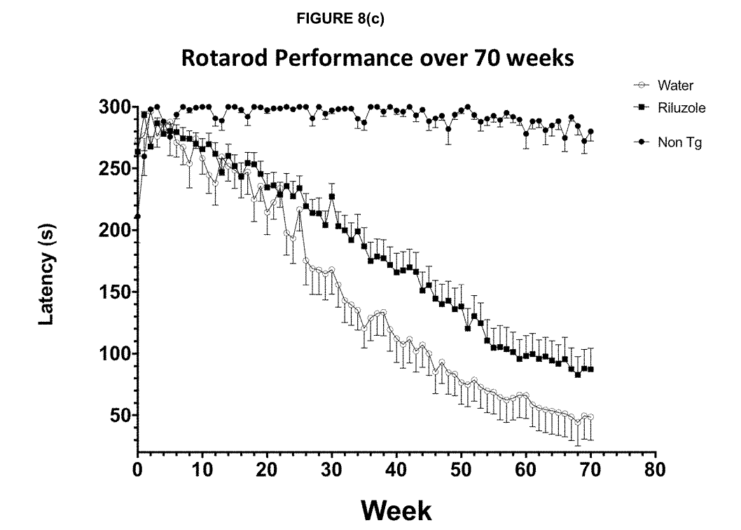

Mice expressing only TDP-43(Q331K) develop a mild but progressive decline in motor function, along with abnormal hind limb splay and tremor. However this mutation does not appear to cause premature death, relative to non-transgenic or TDP-43(WT) animals (see Mitchell et al., supra). In this study this phenotype is manifested as a progressive decline in rotarod latency (FIG. 8(a)). This decline occurs continuously from the start of the study, in contrast to the rotarod performance of the non-transgenic controls which remains steady up to at least one year of age (FIG. 8(a)).

Treatment with tacrolimus delays the progression of motor decline at 0.25 mg/kg, 1.25 mg/kg and 2.5 mg/kg (FIG. 8(b)).

Treatment with 10 mg/kg riluzole causes a similar delay in loss of motor function (FIG. 8(c)). (Note that riluzole is administered in water rather than in the Cremophor/ethanol vehicle used for tacrolimus, so the appropriate control is a group treated with water only.) Therefore the only currently FDA-approved treatment for ALS is also effective in this model.

Previous mouse models expressing mutant TDP-43 have differed substantially in elements of their phenotype from the human disease pathology (see Perrin, 2014, Nature 507(7493):423-5 and Scotter et al., 2015, Neurotherapeutics 12(2):352-63; the disclosures of which are incorporated by reference). However the model used in the current study more faithfully reproduces key aspects of the human disease, including: the late-onset, age-related progressive decline in motor function; cytoplasmic accumulation of insoluble TDP-43 inclusions; motor neuron and cortical neuron loss, accompanied by micro- and astrogliosis; disorganisation of muscle fibres and degeneration of neuromuscular junctions (see Mitchell et al., supra). To our knowledge, this is the first study to show a significant effect of riluzole, the only licensed treatment for ALS, in a TDP-43 mouse model. This lends further support to the validity of this model, and indicates that the positive effect observed for tacrolimus can be extrapolated to the human disease situation.

### EXAMPLE 6

### Effect of Tacrolimus in a Rat 6-OHDA Model of Parkinson's Disease

### Rats

The objective of this study was to investigate the effect of chronic subcutaneous (s.c) tacrolimus treatment in an aged rat model of Parkinson's disease. Lesion of dopaminergic projections was created by unilateral injection of 6-hydroxydopamine (6-OHDA) in the right medial forebrain bundle (MFB), causing a widespread de-afferentation of substantia nigra pars compacta (SNc) and ventral tegmental area (VTA) terminal fields. The animals can then be monitored for amphetamine-induced rotation asymmetry behaviour: because the lesion is unilateral, asymmetric movement is observed. The degree to which a drug treatment is able to counteract the effects of the lesion can be measured by the reduction in the asymmetry of movement (i.e. the number of clockwise (CW) rotations vs. the number of counterclockwise (CCW) rotations).

50 female Fischer rats aged 18 months were stereotactically injected with 4 μl 5.0μg/ml 6-OHDA into the right MFB. After a further 3 weeks, treatment with tacrolimus or vehicle was commenced as follows:
Group 1: 25 rats treated with vehicle (1% Cremophor and 4% ethanol, s.c.) 6 days/week (5 ml/kg until week 18, 2.5 ml/kg thereafter)
Group 2: 25 rats treated with tacrolimus (1 mg/kg, s.c.) 6 days/week (5 ml/kg until week 18, 2.5 ml/kg thereafter)

Treatment was continued until the end-point of the study (approx. 6 months) and behavioural testing was carried out 2 weeks after the 6-OHDA infusion and then bi-monthly at 2, 4 and 6 months. Motor asymmetry was monitored in automated rotometer bowls (TSE Systems, Germany) for 120 minutes after injection of amphetamine (2.5 mg/kg s.c.). The net rotation asymmetry score for amphetamine test was calculated by subtracting contralateral turns from the ipsilateral turns to the lesion side.

After 3 months, 10 randomly selected rats (5 from each group) were terminally anaesthetised. Blood and striatal tissue samples were taken and the posterior brain block containing the SNc was fixed by immersion in 4% paraformaldehyde in 0.1 M phosphate buffer (PB) for 24 hours. Following cryoprotection in 30% sucrose in 0.1M PB for 2-3 days and freezing the blocks in liquid nitrogen, samples were stored at −80° C. for α-synuclein and phospho-Tau (p-Tau) immunohistochemistry (IHC). This was repeated for 11 rats at 6 months (randomly selected from the surviving rats in both groups).

Immunohistochemistry for α-synuclein and p-Tau was carried out. Brain samples containing the SNc region were double-immunostained for p-Tau (AT8) and α-synuclein. The number of positive cells for each single stain and the number of double-stained cells was manually counted for 6 sections per animal.

### Results and Conclusions

### Tacrolimus Reduces Rotational Asymmetry in Unilaterally 6-OHDA-Lesioned Rats

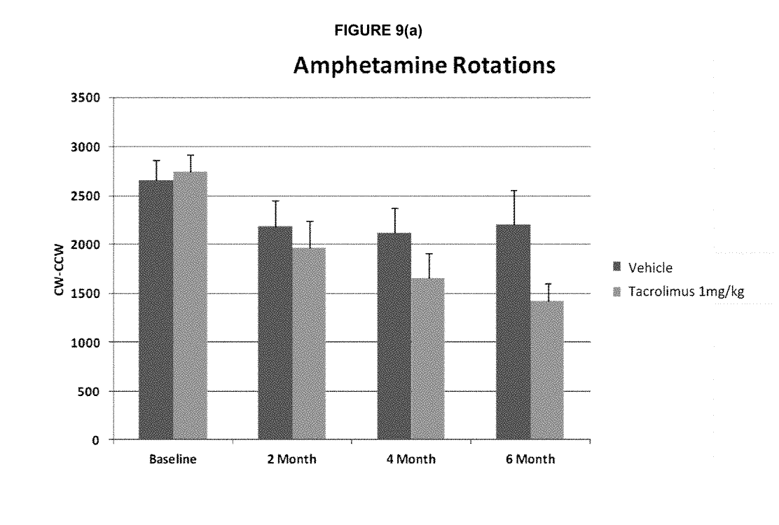

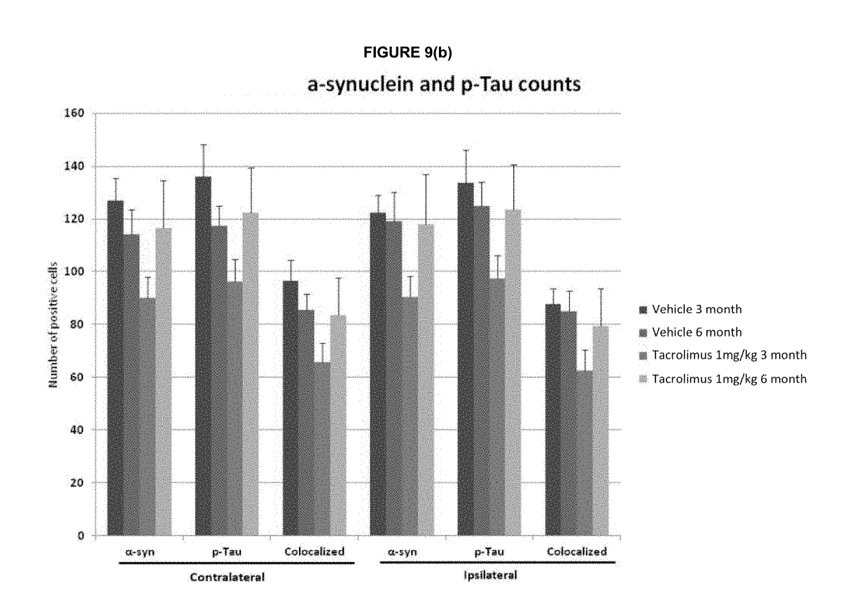

As shown in FIG. 9(a), there was no significant difference in rotational asymmetry between the vehicle and tacrolimus-treated groups prior to the start of treatment, with a CW-CCW value of approximately 2500. When the assay was repeated at the 2, 4 and 6 month time-points the difference between the vehicle and tacrolimus-treated groups became progressively larger, with the tacrolimus-treated rats showing progressively reduced rotational asymmetry. There is a significant reduction in rotational asymmetry in the tacrolimus-treated group by 6 months (compared to the baseline value). In contrast, vehicle-treated rats show no significant change in rotational asymmetry over time. These results suggest that tacrolimus is able to alleviate the extent of the 6-OHDA lesion in this model.

### Tacrolimus Reduces Alpha-Synuclein and Tau Accumulation in the SNpc

FIG. 9(b) shows the results of IHC analysis of the number of cells staining positively for α-synuclein, p-Tau and both. The number of p-Tau, α-synuclein and double positive cells on both sides (contralateral and ipsilateral) remained relatively stable within the treatments. However, the tacrolimus 1 mg/kg 3 months group presented lower counts of positive cells than the rest of the treatment groups (p<0.05, Vehicle vs. tacrolimus 1 mg/kg 3 months), for both p-Tau and α-synuclein. This suggests that tacrolimus may have some effect in reducing (or delaying) p-Tau and α-synuclein accumulation.

### EXAMPLE 7

### Exemplary Unit Dosage Form of the Invention

A hard gelatine capsule was filled with the following composition:

| | Formula- | | Reference |
|---|---|---|---|
| Component | tion (%) | Function | Standard |
| Tacrolimus | 0.30 | Active ingredient | USP |
| Lactose monohydrate | 89.45 | Diluent | Ph Eur |
| Hydroxypropylmethyl | 6.00 | Binder | Ph Eur |
| cellulose | | | |
| Croscarmellose sodium | 4.00 | Super-disintegrant | Ph Eur |
| Magnesium stearate | 0.25 | Lubricant | Ph Eur |
| Ethanol | qs | Binder fluid | |

One capsule as above containing 0.3 mg tacrolimus was administered daily to healthy human volunteers for three successive days. No significant changes in blood TNF-α levels occurred as a result of the administration. Similarly, no change in TNF-α levels occurred as a result of administering 0.6 mg daily of tacrolimus to healthy volunteers. The average trough level of tacrolimus (level after 24 hours of administration of each dose) observed was approximately 220 pg/ml. The average peak level of tacrolimus observed was approximately 3700 pg/mL and the average area under the curve was approximately AUC O_t=23500 (h*pg/ml).

The above capsules may be used to provide the treatments described herein before.

Use of two such capsules simultaneously to provide a single dose of 0.6 mg would be expected to result in a trough level of about 440 pg/ml.

Particular embodiments of the invention are described in the following numbered paragraphs:

Paragraph 1. A method of treating a disease characterised by protein aggregate deposition in neuronal cells which comprises administering to a human in need thereof not more than once a day an effective amount of tacrolimus or a close structural analogue thereof in a dose which does not cause immunosuppression and which produces a trough whole blood level of tacrolimus or its close structural analogue of at least 0.05 ng/mL.

Paragraph 2. Tacrolimus or a close structural analogue thereof for use in the treatment of a disease characterised by protein aggregate deposition in neuronal cells, wherein tacrolimus or its close structural analogue is administered not more than once a day in a dose which does not cause immunosuppression and which produces a trough whole blood level of tacrolimus or its close structural analogue of at least 0.05 ng/mL.

Paragraph 3. The use of tacrolimus or its close structural analogue in the manufacture of a medicament for the treatment of a disease characterised by the deposition of protein aggregates in neuronal cells which medicament contains an amount of tacrolimus or its close structural analogue that when administered once per day does not cause immunosuppression and which has a trough whole blood level of at least 0.05 ng/mL.

Paragraph 4. A method, compound for use or use of a compound as defined in any of paragraphs 1 to 3 wherein the trough whole blood level is at least 0.075 ng/mL, at least 0.2 ng/mL or at least 0.3 ng/mL.

Paragraph 5. A method, compound for use or use of a compound as defined in any of paragraphs 1 to 4 wherein the trough whole blood level is less than 1.2 ng/mL, less than 1.1 ng/mL or less than 1.0 ng/mL.

Paragraph 6. A method, compound for use or use of a compound as defined in any of paragraphs 1 to 5 which employs tacrolimus.

Paragraph 7. A method of treating a disease characterised by protein aggregate deposition in neuronal cells which comprises administering to a human in need thereof not more than once a day an effective amount of tacrolimus or a close structural analogue thereof wherein the dose is from 0.001 mg/kg to 0.02 mg/kg.

Paragraph 8. Tacrolimus or a close structural analogue thereof for use in the treatment of a disease characterised by protein aggregate deposition in neuronal cells wherein tacrolimus or its close structural analogue is administered once a day at a dose of 0.001 mg/kg to 0.02 mg/kg.

Paragraph 9. The use of tacrolimus or a close structural analogue in the manufacture of a medicament for treating a disease characterised by protein aggregate deposition in neuronal cells wherein the medicament contains 0.001 mg/kg to 0.02 mg/kg of tacrolimus or its close structural analogue.

Paragraph 10. A method, compound for use or use of a compound as defined in any of paragraphs 6 to 8 which employs 0.001 mg/kg to 0.02 mg/kg.

Paragraph 11. A method, compound for use or use of a compound as defined in any of paragraphs 7 to 10 which employs not more than 0.013 mg/kg, not more than 0.01 mg/kg, 0.0085 mg/kg or 0.007 mg/kg.

Paragraph 12. A method, compound for use or use of a compound as defined in any of paragraphs 7 to 11 which employs more than 0.0014 mg/kg or 0.002 mg/kg.

Paragraph 13. A method, compound for use or use of a compound as defined in any of paragraphs 7 to 12 which employs 0.0014 mg/kg to 0.0085 mg/kg or 0.002 mg/kg to 0.007 mg/kg.

Paragraph 14. A method, compound for use or use of a compound as defined in any of paragraphs 7 to 13 which employs tacrolimus.

Paragraph 15. A method of treating a disease characterised by protein aggregate deposition in a neuronal cell which comprises administering to a human in need thereof by administration not more than once a day of an effective amount of tacrolimus or a close structural analogue to effect epigenetic modification which leads to an enhancement of autophagy and/or reduction in oxidative stress.

Paragraph 16. Tacrolimus or a close structural analogue thereof for use in the treatment of a disease characterised by protein aggregate deposition by administration not more than once a day of an amount which effects epigenetic modification which leads to an increase in autophagy and/or an improvement in oxidative stress.

Paragraph 17. The use of tacrolimus or close structural analogue thereof in the manufacture of a medicament for the treatment of a disease characterised by protein aggregate deposition in neuronal cells by administration not more than once per day which medicament contains an amount of tacrolimus or close structural analogue thereof which effects epigenetic modification which leads to an increase in autophagy and/or an improvement in oxidative stress.

Paragraph 18. A method, compound for use or use of a compound as defined in any of paragraphs 15 to 17 which employs tacrolimus.

Paragraph 19. A unit dose pharmaceutical composition containing 0.05 mg to 1.3 mg of tacrolimus or a close structural analogue thereof and a pharmaceutically acceptable carrier therefor for use in the treatment of a disease characterised by protein aggregate deposition in neuronal cells.

Paragraph 20. A unit dose pharmaceutical composition as defined in paragraph 19 which does not contain more than 1.2 mg, 0.75 mg, 0.6 mg or 0.4 mg of tacrolimus or a close structural analogue thereof.

Paragraph 21. A unit dose pharmaceutical composition as defined in paragraphs 19 or 20 which comprises not less than 0.06, 0.1 or 0.15 mg.

Paragraph 22. A unit dose pharmaceutical composition as defined in any of paragraphs 19 to 21 adapted for administration by mouth.

Paragraph 23. A method, compound or pharmaceutical composition for use, use of a compound or pharmaceutical composition as defined in any of paragraphs 1 to 22 for use where the disease is amyotrophic lateral sclerosis.

Paragraph 24. A method, compound or pharmaceutical composition for use, or use of a compound or pharmaceutical composition as defined in any of paragraphs 1 to 22, wherein the disease is Alzheimer's disease.

Paragraph 25. A method, compound or pharmaceutical composition for use, or a use of a compound or pharmaceutical composition as defined in any of paragraphs 1 to 22, wherein the disease is Parkinson's disease.

Paragraph 26. A method, compound or pharmaceutical composition for use, or a use of a compound or pharmaceutical composition as defined in any of paragraphs 1 to 22, wherein the disease is Huntington's disease.

Paragraph 27. A method, compound or pharmaceutical composition for use, or a use of a compound or pharmaceutical composition as defined in any of paragraphs 1 to 22, wherein the disease is a synucleinopathy or tauopathy.

Paragraph 28. A method, compound or pharmaceutical composition for use, or use of a compound or pharmaceutical composition as defined in paragraph 27, wherein the disease is a dementia, such as Parkinson's disease dementia and frontotemporal lobe dementia, or other dementias and memory loss conditions associated with age related increase of neurotoxic protein aggregation and/or increased oxidative stress or to defect in autophagy.

Paragraph 29. A method, compound for use or pharmaceutical composition for use, or use of a compound or pharmaceutical composition as defined in any of paragraphs 23 to 28 which employs tacrolimus.

Paragraph 30. A unit dose orally administrable pharmaceutical composition comprising 0.05 mg to 1.2 mg of tacrolimus or a close structural analogue thereof and a pharmaceutically acceptable carrier for use in the treatment of a disease characterised by the deposition of protein aggregates in neuronal cells.

Paragraph 31. A unit dose for use as defined in paragraph 30 wherein the disease is ALS, Parkinson's disease, Alzheimer's disease, Huntington's disease.

Paragraph 32. A unit dose for use as defined in paragraphs 30 or 31 which comprises tacrolimus.

Paragraph 33. A unit dose for use as defined in paragraphs 30 to 32 which comprises not more than 0.9 mg, 0.75 mg, 0.65 mg or not more than 0.5 mg or not more than 0.45 mg of tacrolimus.

Paragraph 34. A unit dose for use as defined in any of paragraphs 30 to 33 which comprises not less than 0.05 mg, not less than 0.1 mg or not less than 0.15 mg of tacrolimus or close structural analogue thereof.

Paragraph 35. A unit dose for use as defined in any of paragraphs 30 to 34 in the form of a tablet or capsule.

Paragraph 36. A method of treatment of a disease characterised by the deposition of protein aggregate in neural cells which comprises administering to a patient in need thereof a pharmaceutical composition by oral administration which comprises an amount of tacrolimus or close structural analogue thereof as set forth in any of paragraphs 30 to 35.

Paragraph 37. A method as defined in paragraph 36 wherein tacrolimus is administered not more than once per day.

1. A method of treating amyotrophic lateral sclerosis or Parkinson's disease which comprises administering to a human in need thereof not more than once a day an effective amount of tacrolimus wherein the dose is from 0.001 mg/kg to 0.02 mg/kg.

2. The method as claimed in claim 1 which employs not more than 0.013 mg/kg, not more than 0.01 mg/kg, not more than 0.0085 mg/kg or not more than 0.007 mg/kg of tacrolimus.

3. The method as claimed in claim 1 which employs more than 0.0014 mg/kg or more than 0.002 mg/kg of tacrolimus.

4. The method as claimed in claim 1 which employs from 0.0014 mg/kg to 0.0085 mg/kg or from 0.002 mg/kg to 0.007 mg/kg of tacrolimus.

5. The method as claimed in claim 1, wherein the disease is amyotrophic lateral sclerosis.

6. The method as claimed in claim 1, wherein the disease is Parkinson's di
